(19) United States
(12) Patent Application Publication
(10) Pub. No.: US 2017/0105813 A1
(43) Pub. Date: Apr. 20, 2017

(54) A ROTATABLE FASTENING DEVICE WITH AN INTEGRAL TORQUE LIMITER

(71) Applicant: MIS Implants Technologies Ltd., MP Misgav (IL)

(72) Inventors: Elad Rash, Beth Lehem Haglilit (IL); Omer Turjeman, Afula (IL)

(21) Appl. No.: 15/128,911

(22) PCT Filed: Mar. 31, 2015
(86) PCT No.: PCT/IL2015/050347

§ 371 Date: Oct. 21, 2016

(30) Foreign Application Priority Data
Apr. 3, 2014 (IL) 231928

(51) Int. Cl. A61C 1/18; A61C 8/00; B25B 23/142; A61B 17/88

(52) U.S. Cl. CPC A61C 1/186; A61B 17/8875; A61C 8/0089; B25B 23/1427; A61B 2090/031

(57) ABSTRACT

a rotatable fastening device with an integral torque limiter that comprises an house component and a coupler assembly wherein the coupler assembly comprises means having at least one springy arm and wherein from the instant of its installation within an inner space of the house component, the springy arm tip is biased to be integrated into a recess of a recesses and protrusions array formed in the house component, and wherein the house component is liable to be actuated by providing a rotatable moment on it, around its lengthwise axis in a manner that as long as the actuated torque being actuated is at a level that does not exceed a pre-set threshold level, the springy arm tip remains integrated within a recess of the recesses and protrusions array of the house component and thus—transfers the torque for turning the 10 coupler assembly concurrently with a rotation of the house component, around the longitudinal axis of the house component and thus—fastening a threadable component to which it is connected; and when the actuated torque is at a level that exceeds the preset threshold level, the springy arm is biased into bending so that its tip is moved unto sinking and is extracted from the recess and pushed unto a sliding movement on the surface area of the array, in a manner that the torque transfer is stopped and the continued rotation of the house component around its lengthwise axis does not lead into a parallel rotation of the coupler assembly and therefore, the fastening of the threadable component is stopped.

### FIELD OF THE INVENTION

The various embodiments described herein generally relate to the field of work tools in general, and particularly to the work tools in the field of those work tools that are used in the field of dentistry, including keys and screwdrivers which the dentist uses in the (inner) space of the mouth, for mounting implants and threading various screws for them.

### BACKGROUND OF THE INVENTION

A preliminary note—the background of the invention, the subject matter of this patent application and the invention itself would be described hereinafter by referring to the accompanying figures and while essentially referring to the dentistry practice, but any professional would understand that the invention is not restricted solely to dental medicine practice.

The dentist is routinely in need of actuating (exerting) an accurate and limited controlled moment in a rotational movement (torque) unto components that serve him in his actions when providing rehabilitant treatments such as, for example—mounting implants, installing restorations (crowns, bridges, prosthesis), surgical treatments of the mouth and jaws (namely Maxillofacial Surgery) and other dentoalveolars treatments.

Considering dental implants: the dental implant actually serves as a kind of an anchoring point on the jaw and it is used in dentistry in order to complete missing teeth arrays in the mouth. One implant or many may support a selection of methods of building teeth arrays, in them a continuum of crowns made of porcelain that is called ‘a bridge’, e. g. extractable implants, extractable prosthetic teeth implants, prosthetic teeth that are threaded directly on the implants and more. It is also feasible to use implants as anchoring for movements of orthodontic teeth and also in restoration (rehabilitation) of faces (for example restoration of ears, nose).

The majority of the dental implants resemble a screw that is threaded into the bone of the jaw. A dental implant is typically a “masonry anchor” having dual (double) threads. The first one is external and serves to anchor the implant to the bone of the jaw, the second one is internal and serves to anchor external accessories unto the implant (restoration assemblies). Actuating a rotational moment (torque) on the implant and on threaded components that are sometimes on it (as a part of the restoration assemblies) has to be accurate and limited in order to avoid excess stress induction on the components (that at certain cases include components that might be cracked due to excess stress on them) as well as on the jaw of the patient that accepts into it the implant that is threaded into it. This and more—variations of implant types, screws and the specific patient are liable to confront the dentist facing challenges of specific compatibility of the rate of the rotational moment that he is required to exert in a given specific case.

Thus, the dentist that is utilizing dental implants is required, as a matter of routine, to exert an accurate and limited moment, and this, it has to be remembered—in a rotational motion that is executed inside the mouth confined space, that naturally renders it difficult for the dentist to provide visual control of the device operated by him to extract the torque at any given instant. As a consequence, for the actuation of said accurate and limited moment imposed on him at that specific operation, the dentist relies chiefly on his senses and expertise—the ‘fruit’ (and outcome) of his experience.

Threading devices that are intended for the dentistry field are known and recognized, including such devices that include a visual indicator providing visual indication as to the given amount of the moment exerted by the dentist at any given instant. See for example—the ratchet device equipped with a pointer (hand) that was described in U.S. Pat. No. 7,597,032, but it was found that because of the limited environmental operation space (inside the mouth space) the dentist is liable to encounter difficulties in forming a line of sight with the indicator, especially over and over for long periods in a continuous availability. This and even more, even when a visual indicator is given, the dentist might fail as per the feedback required from him to prevent operating with an excess torque. The threading device does not include a built in mechanism to prevent erring through exerting excess force by the dentist—and this notwithstanding the visual indication that he received (namely—the device is not a failsafe kind of device).

In addition, there are known and recognized threading devices that are intended for the medical field, inclusive of the dental (dentistry) field, that do include an integral torque limiter, namely devices that in their design they are defined for providing transfer of a rotational moment that does not exceed a pre defined appropriate low measure moment threshold, for example through including a ratchet device. See for example ratchet devices that are described in the following patents—DE 19507535, EP 1110512, U.S. Pat. No. 6,132,435, U.S. Pat. No. 6,398,552 and U.S. Pat. No. 7,025,151; there is a commercial product—ratchet hex driver with torque limiter that is marketed by XIVE Company under the commercial name FRIADENT®. However, it was found that these devices are complicated from the structural point of view and include a relative large number of components, in a manner that it increases their volume and makes it difficult to operate them (conveniently) inside a limited space such as the inner space of the mouth. The complexity of the devices and their closed structures, render it also difficult to rinse them (disinfection), as required from time to time during dental treatments. This and also more—those devices do not provide for mounting them as a replaceable type of module at the end of a universal arm.

Thus, in the period that preceded the invention, the subject matter of this patent application, there existed a need in the field of dental care, for a means that would enable the dentists to actuate an accurate and limited rotational moment on threaded components that he uses inside the mouth space. It is desired that the means would be relatively simple (made up from a limited number of components), easy to be occasionally rinsed and clean, modular in a manner that it would enable suiting it in accordance with the properties of the type of the implant, the screw and the specific patient, and it is also desirable to enable its installation as an easily replaceable module at the end of a tool that serves to actuate the rotational movement. A means that would be made up in compact dimensions as dictated by the geometrical limitations of the work environment (mouth inner space) and limit the moment that is passed by it—and this in a manner that it would be fail safe as said, namely not being dependent on the existence of visual control and on accurate feedback from point of view of the amount of force exerted by the dentist.

### SUMMARY OF THE INVENTION

The invention, the subject matter of this patent application, responds to the need that we pointed at above, through providing a rotatable fastening device with an integral torque limiter, that in its preferred embodiment is given to be mounted as a dismountable and reinstalled anew module at the end of a universal tool that serves for actuating a moment (for example—as a ratchet key for dental applications).

In one aspect of the invention, a rotatable fastening device with an integral torque limiter in accordance with the invention comprises a house component that is formed as a cylindrical sleeve having an inner space with a central axis that extends along its length. An array of alternating recesses and protrusions resembling inner teeth is formed inside the house component wherein it extends around the inner surface of the house inner space on its internal wall. A coupler (or in other words—a clutch) assembly is included inside the house component along the lengthwise axis of the house component. The coupler assembly is being suited to connect with a component amenable to be threaded (hereinafter—threadable) for the sake of fastening it by actuating torque on it. The coupler assembly comprises means having at least one springy arm, wherein from the instant of its installation within the inner space of the house component, the tip of the at least one springy arm is biased to be integrated into a recess of the recesses and protrusions array of the house component.

The house component is liable to be actuated by a rotatable moment on it, around the lengthwise axis of the house component, in a manner that—
a. As long as the actuated torque being actuated is at a level that does not exceed a pre-set threshold level, the tip of the springy arm (one at least), of the means, remains coupled within (inside) a recess of the recesses and protrusions array of the house component and the arm (one at least) transfers the moment for rotating the coupler assembly concurrently with the rotation of the house component, and thus—fastening the threadable component to it is connected.
b. When the actuated torque is at a level that exceeds the preset threshold level, the springy arm (the one at least of the means) is biased into bending such that its tip is moved unto sinking and is extracted from the recess of the recesses and protrusions array of the house component and pushed unto a sliding movement on the surface area of the array, in a manner that the transfer of torque is stopped and the continuation of the rotation of the house component around he lengthwise axis of the house component does not lead into a parallel rotation of the coupler, and in this way the fastening of the threadable component to which it is connected, is stopped.

A rotatable fastening device with an integral torque limiter device in accordance with the invention, is suited to connect (be linked) with an elongated shank component that is liable to be an integral part of the device or a dismountable part of it, namely a shank that is removable and replaceable (through using a connector means that is formed in the coupler assembly) and the end of the elongated shank component that is suited to connect with a component that is threadable for the sake of being fastened by actuating a moment on it is formed as a means from a variety of means that are known in the discussed field (for example—a tip key, a tip of an hexagonal screws key 0.05″, an implants hexagonal 2.45 mm key, a specific designated key (for example—a conical connections key) for implants of a selected make or another, in a narrow or a wide platform, a screwdriver tip, or a socket spanner end).

In a preferred embodiment of the invention, the means with at least one springy arm is advanced to a multi springy arms means wherein the plurality of springy arms are deployed in a circumferential array while they are protruding from the lengthwise axis of the house component, and their tips are biased to be integrated (coupled) in a number (plurality) of recesses of the recesses and protrusions array of the house component (in accordance with the number of arms).

A rotatable fastening device with an integral torque limiter in accordance with the invention, is assembled from a relatively small number of parts (three to five, according to the selected embodiment) and enables an easy rinsing run of the coupler assembly (that can be exposed or at least accessible by removing a screwed cover, subject to the selected embodiment).

By another and additional aspect of the invention, a rotatable fastening device with an integral torque limiter in accordance with the invention, is formed with an house component whose external surface areas include means that enable connectivity of the device as a replaceable module, with a ratchet key as needed for actuating the moment on the device by it.

Still other aspects, embodiments, and advantages of these exemplary aspects and embodiments are discussed in detail below. Embodiments disclosed herein may be combined with other embodiments in any manner consistent with at least one of the principles disclosed herein, and references to “an embodiment or the like are not necessarily mutually exclusive and are intended to indicate that a particular feature, structure, or characteristic described may be included in at least one embodiment. The appearances of such terms herein are not necessarily all referring to the same embodiment.

It is to be understood that both the foregoing general description and the following detailed description are exemplary and explanatory and are intended to provide further explanations of the invention as claimed.

### BRIEF DESCRIPTION OF THE ACCOMPANYING FIGURES

Various aspects of at least one embodiment are discussed below with reference to the accompanying figures, which are not intended to be drawn to scale. The figures are included to provide illustration and a further understanding of the various aspects and embodiments, and are incorporated in and constitute a part of this specification, but are not intended as a definition of the limits of the invention. In the figures, each identical or nearly identical component that is illustrated in various figures is represented by a like numeral. For purposes of clarity, not every component may be labeled in every figure.

FIG. 1 constitutes a view in perspective of one example, first embodiment, of a device in accordance with the invention.

FIGS. 2 and 3 constitute exploded views in perspective from two different angles of the components of the device that is illustrated in FIG. 1.

FIG. 4a to FIG. 4c constitute, each one of them, couples of cross section figures, in accordance with the cross section views that are marked a-a and b-b in FIG. 1, in a manner that depicts the various modes of operation of the integrated torque limiter of the device that is illustrated in FIG. 1 to FIG. 3.

FIG. 5 constitutes an enlarged local cross section view in accordance with the sector that is marked c-c in FIG. 4a that displays the manner of assembling the components of the device that is illustrated in FIG. 1 to FIG. 3 and FIG. 4a to FIG. 4c and the manner of disassembling them.

FIGS. 6 and 7 constitute illustrations of usage for dental work of a device a like the one that is illustrated in FIGS. 1 to 5. FIG. 6 depicts operating the device by direct manual operation (holding it by hand) and FIG. 7—through using a universal ratchet key that enables installation of a variety of devices in accordance with the invention, as an exchangeable module at the end of such universal (unity) single key.

FIG. 8 constitutes a view in perspective from two different angles, of another example of a different configuration, second embodiment, of a device in accordance with the invention—a device without an elongated integral shank component in it, but with a connector means that enables installation on it in a manner that is given to disassembling, removal and changing of variety of elongated shank components.

FIGS. 9 and 10 constitute exploded views in perspective from two different angles of the components of the device illustrated in FIG. 8.

FIG. 11 constitutes a view in perspective of an example of a different configuration, third embodiment, of a device in accordance with the invention—a device equipped with a threadable cover component and a coupler assembly which is extractable by sliding out from the house component.

FIGS. 12 and 13 constitute exploded views in perspective from two different angles of the components of the device that is illustrated in FIG. 11.

FIGS. 14 and 15 constitute exploded views in perspective from two different angles of the components of the device that was illustrated in FIG. 11 to 13 following opening of the cover component and extracting the coupler assembly from the house component (by extraction and sliding it from the house component); for example—in order to be rinsed).

FIG. 16 constitutes a view in perspective from two different angles (one with a partial cross section view) of another example of a different configuration, fourth embodiment, of a device in accordance with the invention—a device that is also equipped with a threadable cover component and a coupler assembly which is extractable by sliding it out from the house component, but without an integral elongated shank component on it, but rather with a connector means that enables installation on it in a manner that it enables disassembling removal, and exchanging of variety of elongated shank components.

FIGS. 17 and 18 constitute exploded views in perspective from two different angles of the components of the device that is illustrated in FIG. 16.

### DETAILED DESCRIPTION OF A PREFERRED EMBODIMENTS OF THE INVENTION

Various devices will be described below to provide an example of an embodiment of each claimed invention. No embodiment described below limits any claimed invention and any claimed invention may cover devices that differ from those described below. The claimed inventions are not limited to devices having all of the features of any one device described below or to features common to multiple or all of the devices described below. It is possible that a device described below is not an embodiment of any claimed invention. Any invention disclosed in a device described below that is not claimed in this document may be the subject matter of another protective device, for example, a continuing patent application, and the applicants, inventors or owners do not intend to abandon, disclaim or dedicate to the public any such invention by its disclosure in this document.

Furthermore, it will be appreciated that for simplicity and clarity of illustration, where considered appropriate, reference numerals may be repeated among the figures to indicate corresponding or analogous elements, in addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

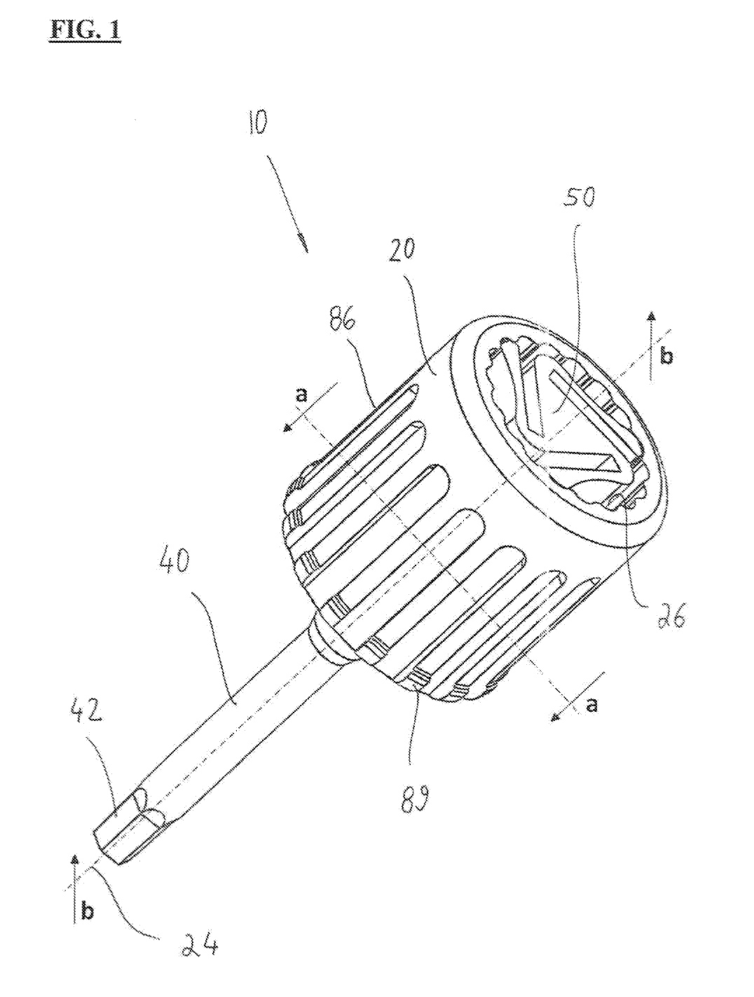

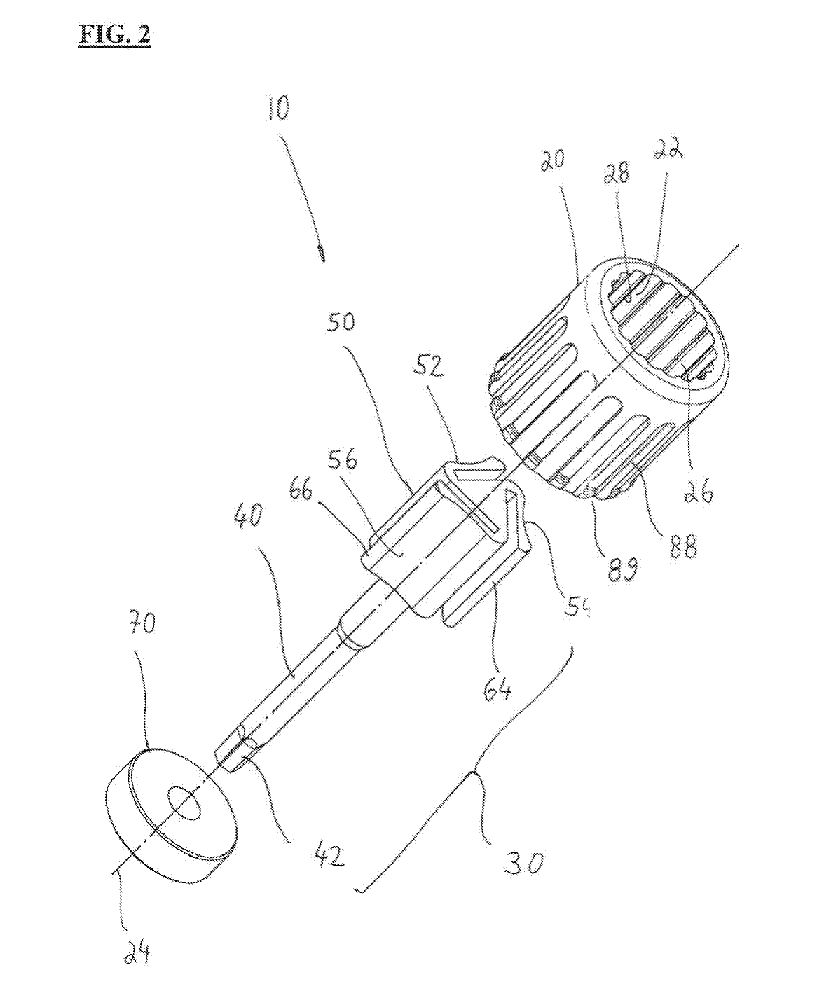

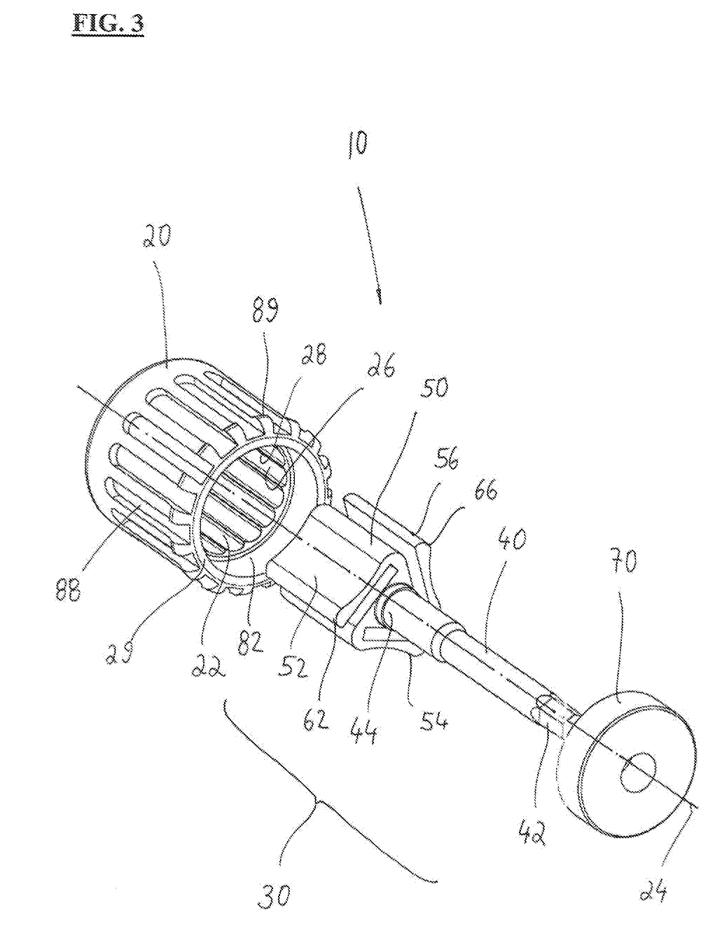

Reference is made to FIGS. 1 to 3. FIG. 1 constitutes a view in perspective of an example device 10 in accordance with the invention. FIGS. 2 and 3 constitutes exploded views in perspective from two different angles of the components of device 10.

Device 10 comprises a house component 20 that is formed as a cylindrical sleeve having inner space 22 with a central axis 24 that extends along it. An array 26 of alternating recesses and protrusions, resembling inner teeth is formed inside house component 20 wherein it extends around the circumference of its inner space 22 on the sleeve inner wall 28.

Coupler assembly 30 is suited to be included within house component 20 along its central axis 24 and is suited to be connected with a threadable component (that is not illustrated) for the sake of fastening it by actuation of a torque on it. In the illustrated example, coupler assembly 30 comprises—
a. An elongated shank component 40 that is suited to be installed inside the inner space of house component 20, wherein its one end 42, the end that is suited to be connected with a threadable component (that is not illustrated) for fastening by actuation of a rotatable moment on it, protrudes from house component 20.
b. Means 50, that in the illustrated example is a means with multi springy arms, in the illustrated example—three springy arms 52, 54 and 56, that is suited to be mounted on the second end—44 of elongated component 40, wherein from the instant of installing elongated component 40 inside inner space 22 of house component 20, the springy arms—in the illustrated example—52, 54 and 56, extend in a circumferential array wherein they protrude from lengthwise axis 24 of house component 20, and their tips (in the illustrated example)—62, 64 and 66, are biased to integrate inside recesses of the recesses and protrusion array 26 of component 20.
c. Disc 70. From the instant of assembling the device, disc 70 is positioned alongside the springy multi armed means 50. Disc 70 is suited for being affixed (for example by press-fit, bonding or by any other assembling means), on a segment of elongated shank 40. Concurrently, house component 20 is formed at one end of it—29, with bracket 82 that is formed inside house component 20 wherein it extends around the circumference of its inner space on its inner wall 28. From the instant of assembling the device, the surface area of bracket 82 is suited to be a bearing disc 70 for rotating around the lengthwise axis 24, together with means 50.

Any professional would understand that in accordance with the illustrated example, house component 20 is formed wherein its inner space 22 is formed as a cylindrical sleeve that is open on its two sides, therefore—from the instant of assembling device 10, dismantling disc component 70 is not necessary in order to provide access and rinsing the inside of the device (see in FIG. 1—exposing the multi springy arms means 50). Especially as it is feasible to form disc 70 with ‘passing through’ openings in it (that are not illustrated) in a manner that would enable passage of the rinsing liquid from one side to the other, through house component 20 inner space.

In the illustrated example, elongated shank component 40 is formed (for example—by turning from stainless steel PH 455) as an axis having a typical round cross section. End 42 of elongated shank component 40, the end that is suited to connect with a threadable component (that is not illustrated) for the sake of fastening it by actuating a torque on it, is formed as an end of a hexagonal Allen key. The discussed subject is, as said, only an example and any professional would understand that the means can be another and different means. for example tip key, end of a hexagonal screws key 0.05″, an implants hexagonal 2.45 mm key, a specific designated key (for example—a conic connections key) for implants of a selected make or another, in a narrow or a wide platform, a screwdriver head or an end formed as a socket spanner).

In the illustrated example, means 50 is manufactured as one homogenous part with the elongated shank (as said, for example, from stainless steel PH 455 and by machining it utilizing electro erosion technology) but any professional would understand that under the same approach, means 50 is liable to be manufactured as a separate part and affixed to the end of elongated shank component 40 (for example—by press-fit or adhesive).

As said, in the illustrated example, means 50 is a multi springy arms means, in the illustrated example—means that is formed with three springy arms 52, 54 and 56, that are located in an equal distribution around—and in relation to the lengthwise axis 24 (manufactured for example—by electro erosion technology). Each one of the springy arms is formed as a kind of a spring leaf or a flexible “wing”, in the illustrated example—in a rectangular configuration. Each one of the leaf springs extends in an essentially tangential direction in relation to lengthwise axis 24, as an outstretched beam wherein it is harnessed to a one end while the other end (tips 62, 64 and 66) is free and located at the tip of a forearm sector that can be bent in a flexible movement around the opposing harnessed end.

Professionals in this field would understand that solely an example is being treated, and that the means might be formed with a different number of springy arms, inclusive—one with only a single arm, or with one arm as said or more, that can be formed in a different configuration (a configuration that is not necessarily rectangular).

House component 20 is liable to be manufactured from stainless steel SS 420 by turning technology and then milling or processing by an electro erosion run. Disc component 70 is liable to be manufactured from stainless steel SS 303.

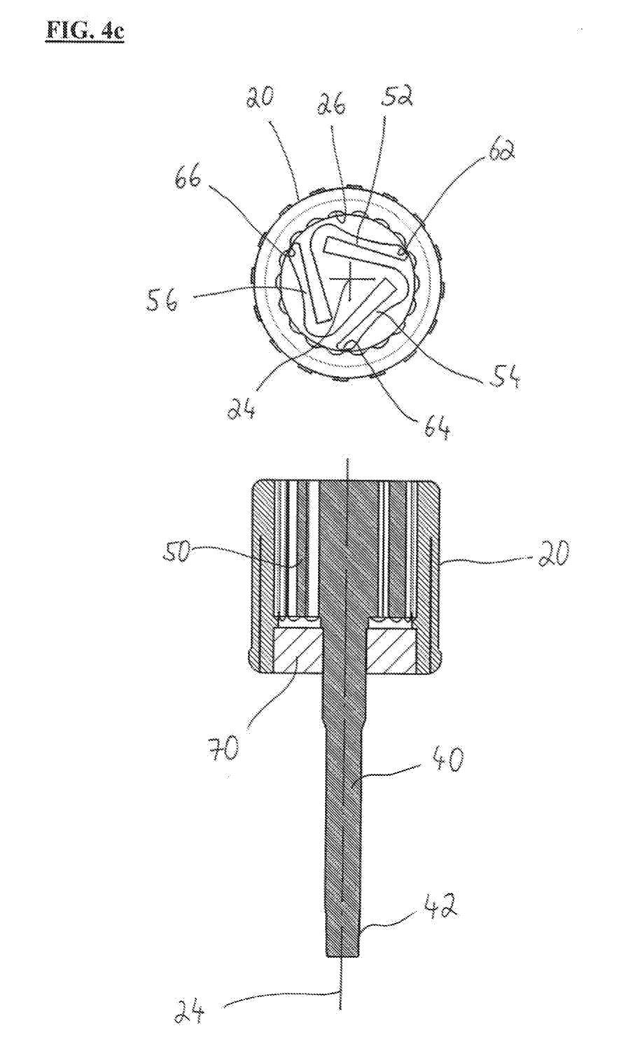

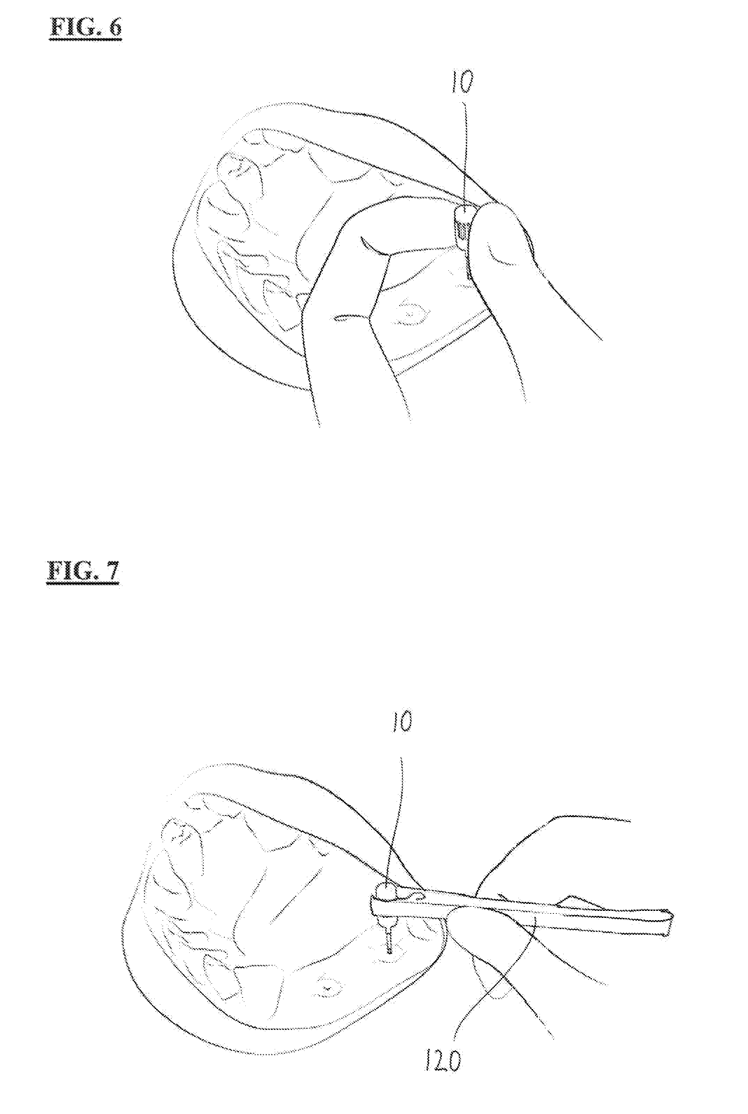

House component 20 is operable by applying a rotational momentum (torque) on it and around the lengthwise axis 24 of house component 20. In the illustrated example, the external surface area 86 of house component 20 is formed with means that enable linking of the device as an exchangeable module, with a ratchet key to be used for actuating a torque on the device. In the illustrated example—an array 88 of longitudinal recesses and protrusions that extend alternatively over at least of a sector of house component 20 and around its circumference. In addition, a circumferential shoulder 89 is formed alongside array 88 of longitudinal recesses and protrusions wherein it protrudes from the house component 20 for preventing sliding of the ratchet key from the instant of connecting with the array. Naturally, array 88 also facilitates manual holding of device 10 and direct actuation of said torque on it (and sees below in the continuation when referring to FIGS. 6 and 7).

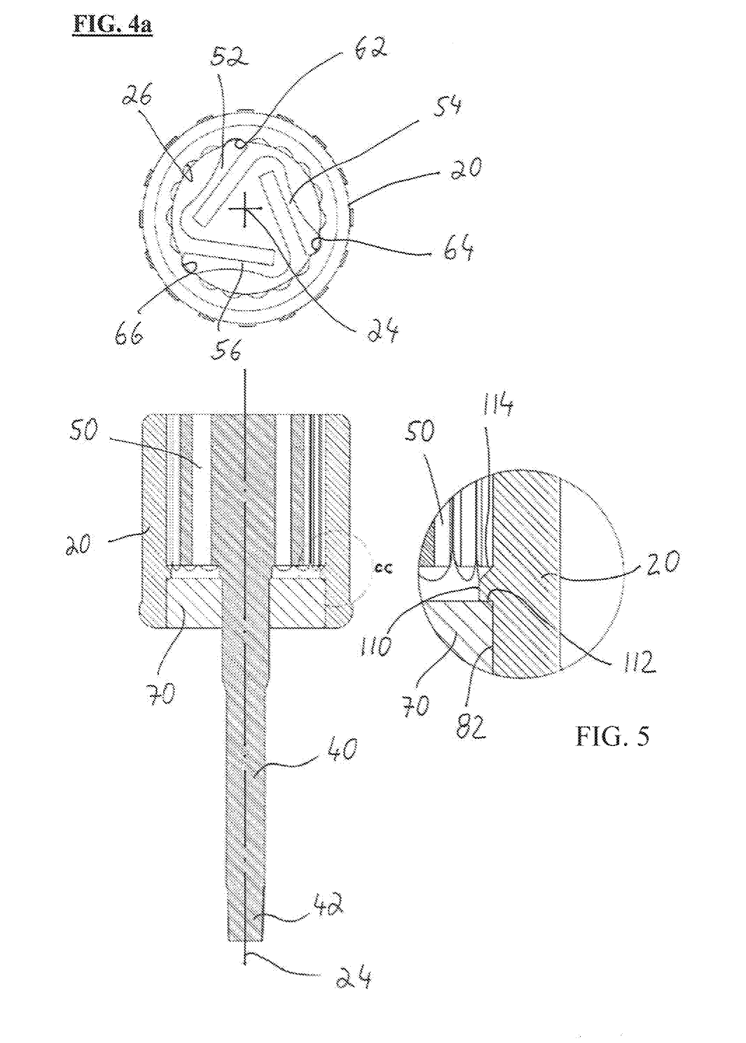

For understanding the manner of operation of the coupler assembly 30 in device 10, let's refer to FIG. 4a to FIG. 4c. FIGS. 4a to 4c constitute couples of cross section figures, in accordance with the cross sections that are marked a-a and b-b—each one of them in FIG. 1, in a manner that depicts the various operational states of the integral torque limiter of device 10.

FIG. 4a displays the operational state at instants of fastening (or releasing) a threadable component (that is not illustrated). At this state, the moment being actuated on house component 20 is at a value that is not in excess of a preselected threshold value. At this state, the arms tips (in the illustrated example—tips 62, 64 and 66), are left integrated inside recesses of the recesses and protrusions array 26 of house component 20. Arms 52, 54 and 56 transfer the moment for rotating elongated shank component 40 concurrently with rotating house component 20 around the longitudinal axis 24 of house component 20 and thus—fastening (or releasing) the threadable component (that is not illustrated), (the component unto which elongated shank component 40 is connected at its end 42).

FIGS. 4b and 4c display the operational state wherein the actuated moment is at a level that exceeds the pre-set threshold value. At the state that is displayed in FIG. 4b, arms 52, 54 and 56 are biased into bending, in a manner that their tips, tips 62, 64 and 66, are pushed unto sinking and start being extracted from the recesses of the recesses and protrusions array 26 of house component 20. At the state that is displayed in FIG. 4c, tips 62, 64 and 66 are pushed into a sliding movement along the area surface of array 26, in a manner that the transfer of moment terminates and the continuation of rotation of house component 20 around longitudinal axis 24 of house component 20 does not (any longer) leads to corresponding rotation of elongated shank component 40, and thus ends the fastening of the threadable component (that is not illustrated), (the component unto which elongated shank component 40 is connected at its end 42).

In the illustrated example, the recesses and protrusions array 26 of house component 20 is formed with recesses endowed with a roundish typical cross section and tips 62, 64 and 66 of the springy arms are also formed accordingly with roundish ends meant to integrate into the recesses of the house component. More in the illustrated example, the three springy arms 92, 94 and 96 are formed, as said, as a kind of a springy leaf with a rectangular configuration (see FIGS. 2 and 3), having a typical cross section of a roundish segment on the one side and a flat segment on its second side. Any professional would understand, that solely an example is being discussed—and the array of recesses the intermingling tips and the cross section of every one of the springy arms is liable to be different.

Design of a coupler as was described above is within the scope of knowledge of professionals in the field. Professionals that are experienced in weighing for this end the data in regard of the dimensions of relevant contact planes, angles, friction coefficients, the bias force applied by the springy means, and the thresholds of the rotational moments (torques) that is to be transferred before the occurring of extraction and sliding. For designing a suitable coupler, it is expedient (possible) to use CAD (Computer Aided Design) software—for example SolidWorks® in order to calculate the stresses that are being developed in light of the required shifting of the springy arms (the sinking of the springy arms), in order to provide for the extraction and sliding the mechanisms.

Thus, any professional would also understand that the structure of the coupler as was described above while referring to FIGS. 4a to 4c is solely an example for a coupler that can be implemented in a device in accordance with the invention.

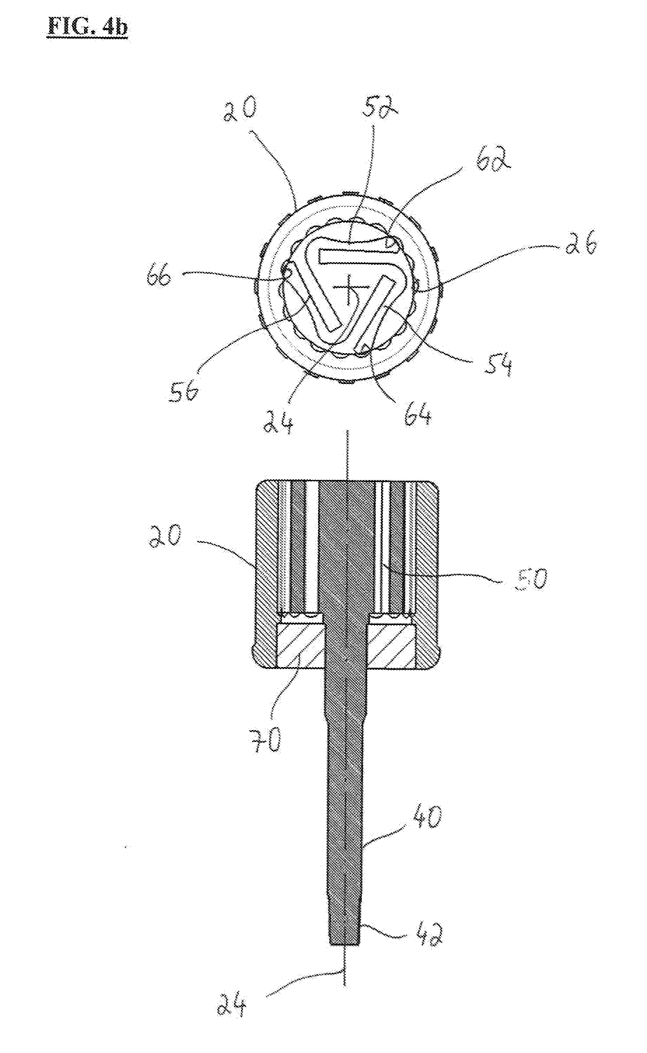

Reference is additionally given to FIG. 5. FIG. 5 constitutes an enlarged limited local cross section in accordance with the portion that was marked c-c in FIG. 4a, depicting the manner of assembling the components of device 10 that is illustrated in FIGS. 1 to 3 and in 4a to 4c and their disassembly procedure.

In the illustrated example, house component 20 is formed with an intermediate segment 110 that is formed inside house component 20 wherein it extends circumferentially around its inner space 22 while it protrudes from its inner surface 28 and separates between the alternating recesses and protrusions array 26 and bracket 82.

Assembling device 10 requires pushing means 50 (the multi springy arms unit) by exerting force, from the direction of bracket 82 to movement in the one direction—along the lengthwise axis 24 of house component 20 towards and beyond intermediate segment 110, and until the surface area of disk 70 (that as said is affixed to elongated shank component 40) is coming into contact with one side of intermediate segment 110 (in a manner that prevents the extraction of the coupler assembly from the one side of the house component 20).

Disassembly of device 10 necessitates the pushing of means 50 (the multi springy arms means) also by using force, from the direction of the alternating recesses and protrusions array 26, into movement in the other (opposite) direction—along longitudinal axis 24 of house component 20, to the other side of the intermediate segment 110, through and while passing over intermediate segment 110 and extracting disc 70 from bracket 82 (exerting force that usually prevents the extraction of the coupler assembly from the second side of house component 20). In the illustrated example, intermediate segment 110, is formed on the one side that is connected with the surface area of disk 70, as a protruding shoulder 112 and on its second side, the side is connected with means 50 (the multi springy arms unit), as a slanted depression 114. Thus, in a device 10 in accordance with the illustrated example, it is required to exert relatively high level force along lengthwise axis 24 for assembling the device or disassembling it.

Reference is made to FIGS. 6 and 7. FIGS. 6 and 7 constitute illustrations of usage for dental work of device 10. FIG. 6 depict operating the device by direct manual operation (holding it by hand) and FIG. 7—operating device 10 while utilizing a universal ratchet key 120. In the illustrated example—a ratchet key whose external shape is the subject matter of a Design application IL 54515 by the applicant of the present patent application. Any professional would understand that it is feasible to install a variety of devices according to the invention, as a changeable module at the end of such universal key. In other words—a variety of devices in accordance with the invention, that are liable to be suited—each one, to transferring torque at a different value (moment's force) to a variety of threadable fastened components, are liable to be mounted by the dentists as changeable modules, at the end of a key, ratchet or other, which is a universal (unity) single key.

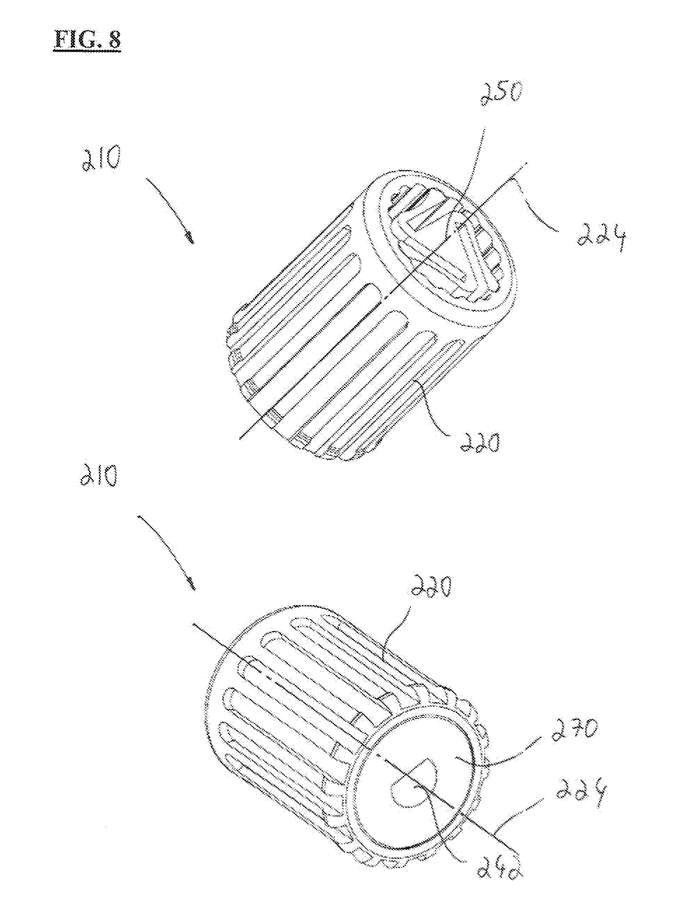

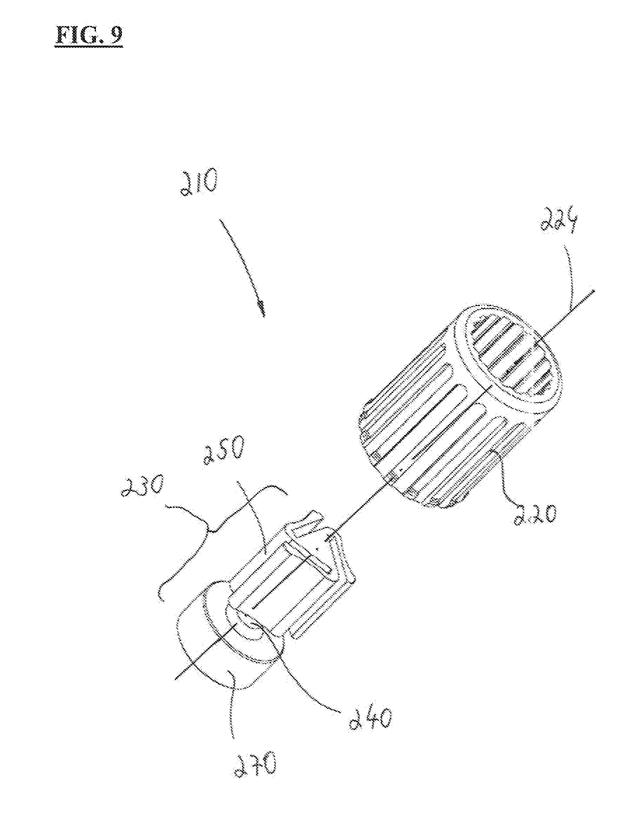

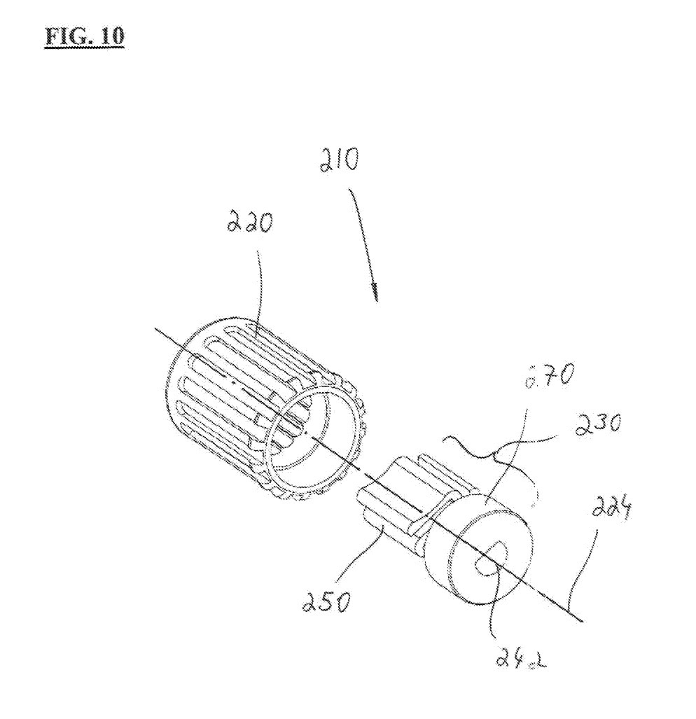

Reference is given to FIG. 8 to 10. FIG. 8 constitutes a view in perspective from two different angles, of another example of a different configuration, second embodiment, 210, of an device in accordance with the invention—a device without an elongated integral shank component in it but with a connector means 242 that enables installation on it of a variety of elongated shank components (that are not illustrated), in a manner that provide for disassembling, removal and replacing of them. Elongated shank components that are suited, on their side, each one, to connect with a threadable component (that is not illustrated) for the sake of fastening it by exerting a torque on it. FIGS. 9 and 10 constitute exploded views in perspective from two different angles of the components of device 210.

Similarly to device 10 that was described above when referring to FIGS. 1 to 7, also device 210 comprises similar house component 220 and coupler assembly 230. But to distinguish from the above cited device 10, in device 210 the disc component 270 on its one side is connected to the multi springy arms means 250 through a connecting stem 240 that extends along the longitudinal axis 224.

In the illustrated example, disk component 270, connecting stem 240 and the multi springy arms means 250 are formed as one unified homogenous part (but any professional would understand that only an example is talked about and it is feasible to manufacture the coupler assembly from separate parts that will be affixed one to the other).

On its second side, disc component 270 is formed with connector means 242 that enables installing on it—in a disassembling possibility, removal and replacement of elongated shank components (that are not illustrated), that are suited, on their side, each one, to connect with a threadable component (that is not illustrated) for the sake of fastening it by exerting a torque on it (for example—shanks whose ends are formed as a means that consists means from between a (group) variety of means that includes—a tip key, an end of an hexagonal screws key 0.05″, an implants hexagonal 2.45 mm key, a specific designated key (for example—a conic connections key) for implants of a selected make or another, in a narrow or a wide platform, a screwdriver head, and a socket spanner end). In the illustrated example, connector means 242 consists a connector adapted to ISO 1797-1 (Dentistry—Shanks for Rotary Devices), but any professional would understand that it is referring solely to an example and other connectors can be used as well.

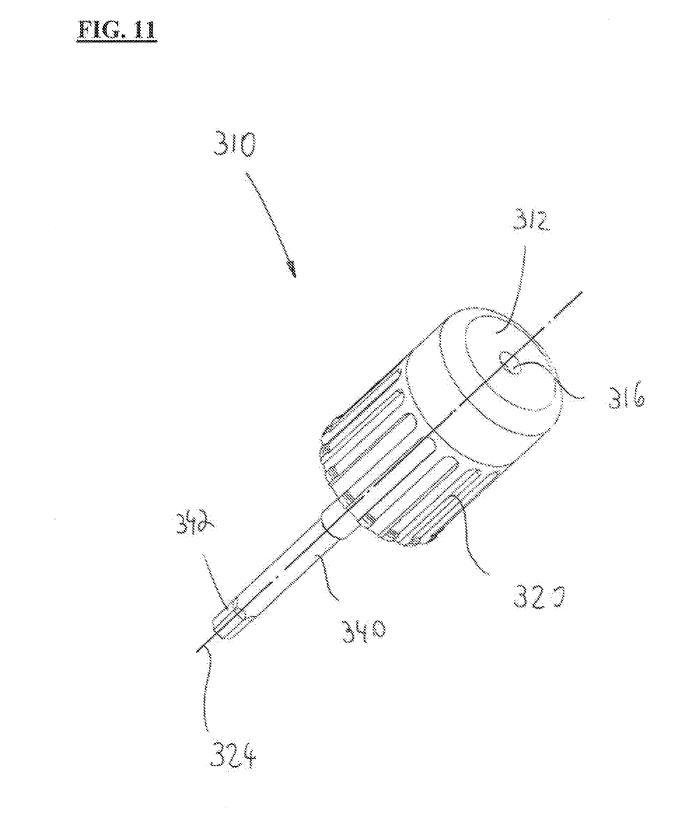

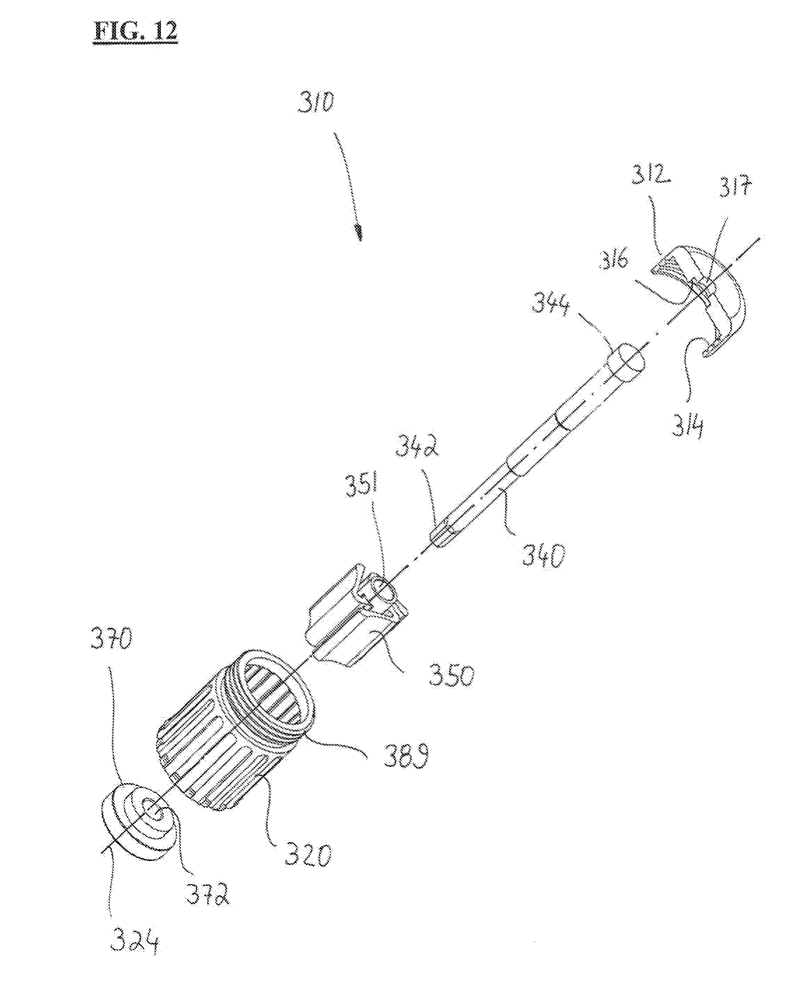

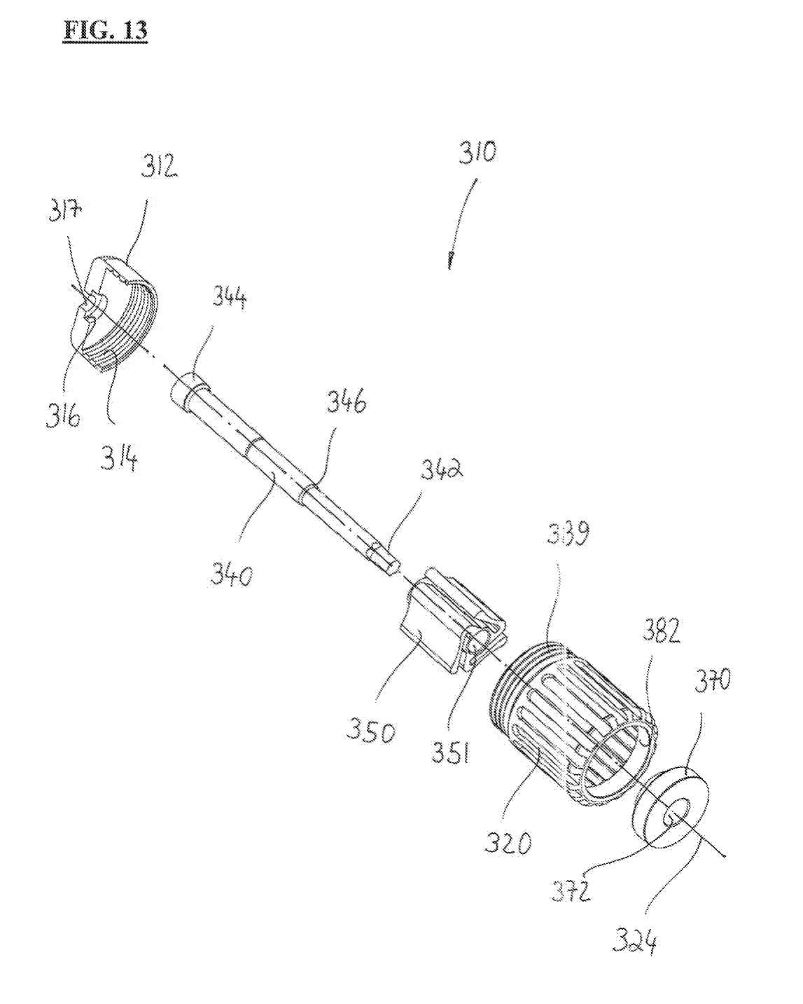

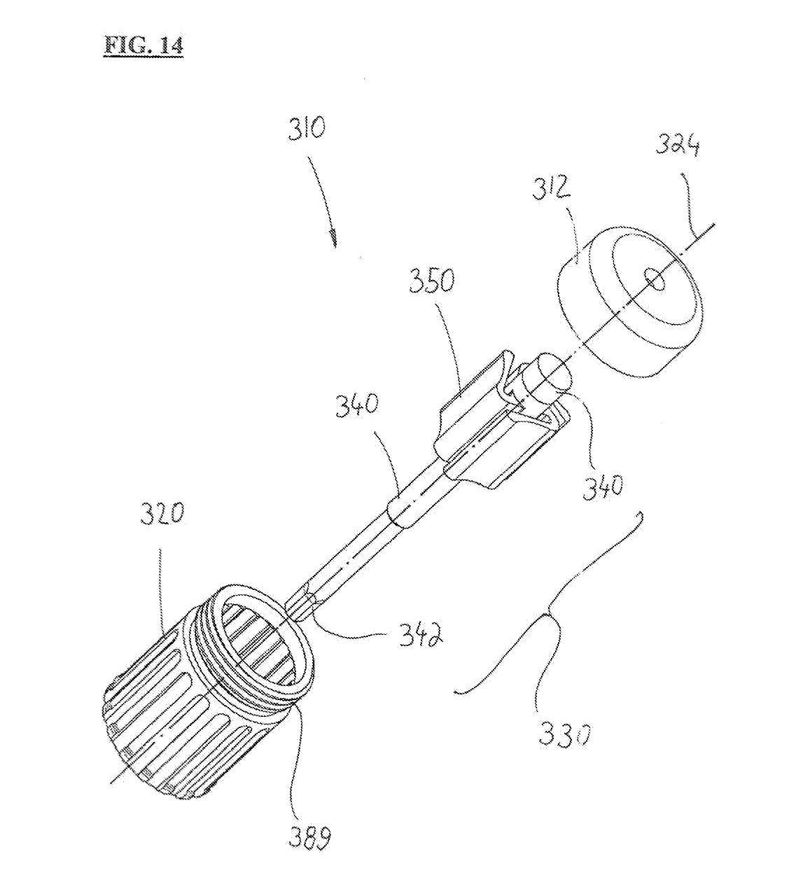

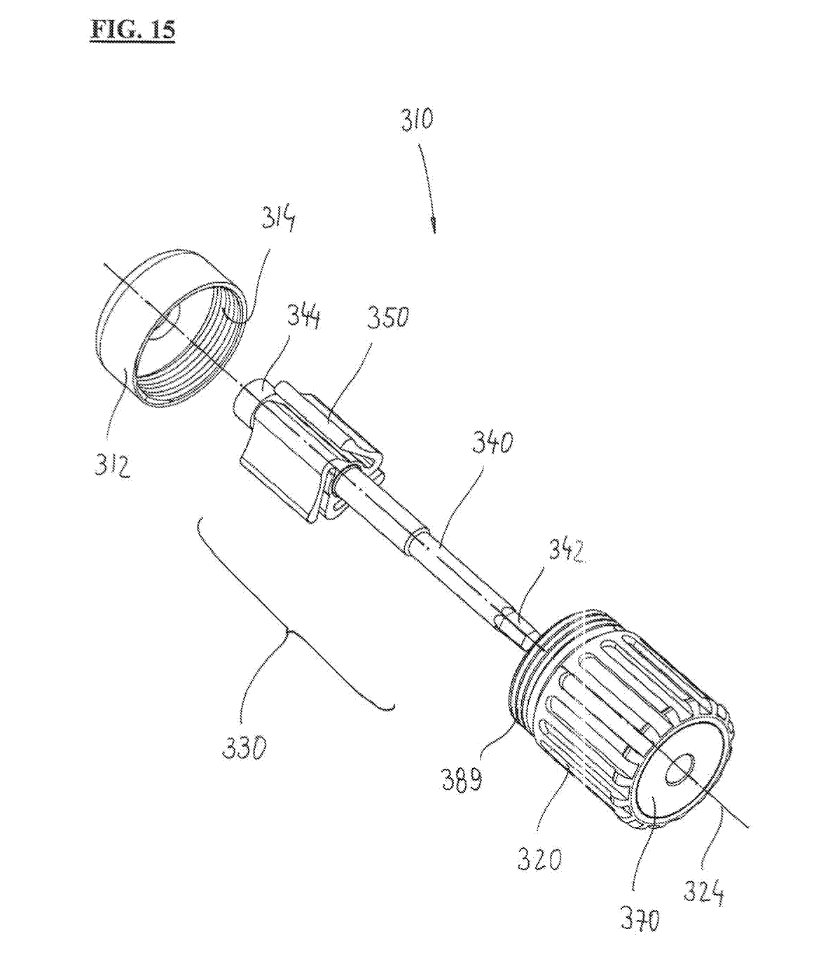

Reference is made to FIG. 11 to 15. FIG. 11 constitutes a view in perspective of an example of a different configuration, third embodiment, 310, of a device in accordance with the invention—a device equipped with a threadable cover component 312 and coupler assembly 330 that is extractable and given to be extracted by sliding it out from the house component 320. FIGS. 12 and 13 are exploded views in perspective from two different angles of the components of device 310. FIGS. 14 and 15 are exploded views in perspective from two different angles of the components of device 310 after opening cover component 312 and extracting coupler assembly 330 from house component 320 (extracting it and sliding it from house component 320), (for example for the purpose of rinsing it).

Similarly to devices 10 and 210 that were described above when referring to FIGS. 1-8 and 9-10, respectively, also in device 310 its house component 320 is formed in one end of it, with bracket 382 that is formed inside house component 320 wherein it extends around the circumference of its inner space on its inner wall (but as we will see hereinafter, does not serve as a bearing to the external surface area of a disc to turn, but rather affixes the disc outer surface to itself). Concurrently, and yet different from the houses components of devices 10 and 210, house component 320 is formed at its second end with an external threaded segment 389 that is formed on a portion of the outer surface of the house component.

Device 310 comprises a disc component 370 endowed with central opening 372. Disc component 370 is suited to be affixed inside bracket 382 that is formed within house component 320 and to provide bearing to coupler assembly 330 for turning coupler assembly 330 around the lengthwise axis 324 through the inner surface area of opening 372 of disc 370 (to distinguish and be different from devices 10 and 210 in which the bearing function is accomplished on the outer surface area of the disc components wherein at those embodiments of those outer surface area of the disc component are not affixed to the houses components but rather turn together with the couplers assemblies and as a part of them).

As mentioned hereinabove, device 310 comprises in addition, a cover component 312. Cover component 312 is formed with an internal thread portion 314 that matches an external thread portion 389 of house component 320. Cover component 312 is formed also with a central opening 316. Cover component 312 is suited to be mounted with a thread, fastening it to house component 320 and to provide also, bearing to coupler assembly 330 (in addition, as said, to the bearing provided by the inner opening 372 of disc 370) for turning coupler assembly 330 around longitudinal axis 324. Cover component 312 is bearing coupler assembly 330 through inner surface area that is formed as a shoulder 316 around central opening 317.

In device 310, coupler assembly 330 is formed with an elongated shank component 340 that protrudes on the two sides of multi springy arms means 350. One end —342 of elongated shank component 340 is suited to connect with the threadable component (not illustrated) for (the need of) fastening it by exerting a torque on it.

From the instant of installing multi springy arms means 350 of assembly 330 within the inner space of house component 320, the end of shank 342 protrudes from the house component 320 (see FIG. 11).

From the instant of assembling device 310, a segment of elongated shank component 340 that is located between the multi springy arms means 350 to the one end 342 of the elongated shank, is suited to connect for contact with the inner surface area of the central opening 372 of disc component 370 (that is affixed to house component 320) and thus, the inner surface area of central opening 372 of disc component 370 provide a bearing to coupler assembly 330.

Second end 344 of elongated shank component 340 is suited to contact with the inner surface area of shoulder 316 that is formed around the central opening 317 of cover component 312 and thus, as said, the inner surface area of shoulder 316 of cover component 312 acts also as bearings of coupler assembly 330.

In the illustrated example, elongated shank component 340 and means 350 with multi springy arms are two separate parts that are affixed on to the other (for example—by press fit and or adhesive or any other known assemblage means). For this end, means 350 multi springy arms means 350 is formed with a passing through opening 351 that is suited to accept and be affixed in it the elongated shank 340 wherein it protrudes on its two sides (any professional would understand that we are considering solely an example, and the components are also liable to be manufactured as a homogenous single part).

In the illustrated example, elongated shank 340 is formed with a protruding shoulder 346 that is suited to connect unto the surface area of disc component 370 in a manner that prevents sliding of the shank by moving along axis 324 in one direction—through opening 372 of the disc. Simultaneously, cover component 312 is formed with shoulder 316 (see local cross sections in FIGS. 12 and 13). Shoulder 316 is formed as it protrudes from the inner surface area of central opening 317 of cover component 312. Thus, shoulder 316 is suited to connect for contact with end 344 of elongated shank 340 in a manner that prevents the sliding of the shank in motion along axis 324 in the second direction—through central opening 317 of cover component 312.

Thus, any professional would understand that in contradistinction with devices 10 and 210 that were described above while referring to FIGS. 1-8 and 9-10, respectively, in device 310, including the coupler assembly within the house component, as well as dismantling it and extracting the coupler assembly from it, do not require substantial force, and at the time of assembling or disassembling (for example for rinsing purpose) the coupler assembly might slide inwards or outwards along the longitudinal axis with a relative ease and as a one (single) unit (see FIGS. 14 and 15). This is practically all that is required from the echelon that is using the device according to this embodiment of the invention (the dentist or his assistant), as for maintenance for the device. Concurrently, any professional would understand that in device 310 (different from the case of devices 10 and 210), for cleaning the assembly, it is required first to dismantle a threadable cover component.

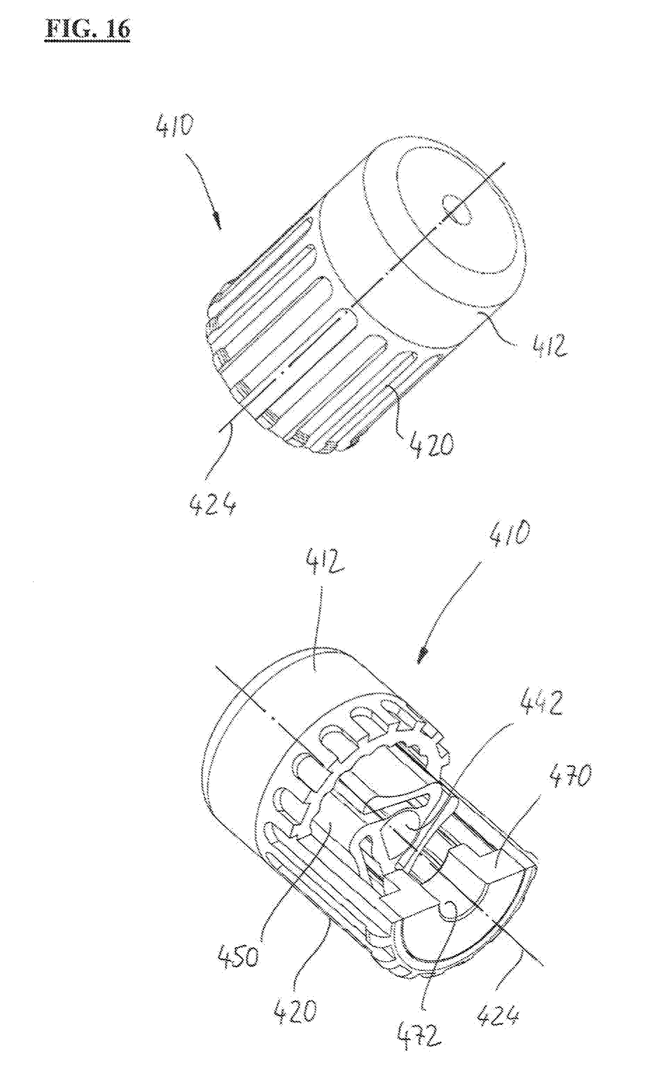

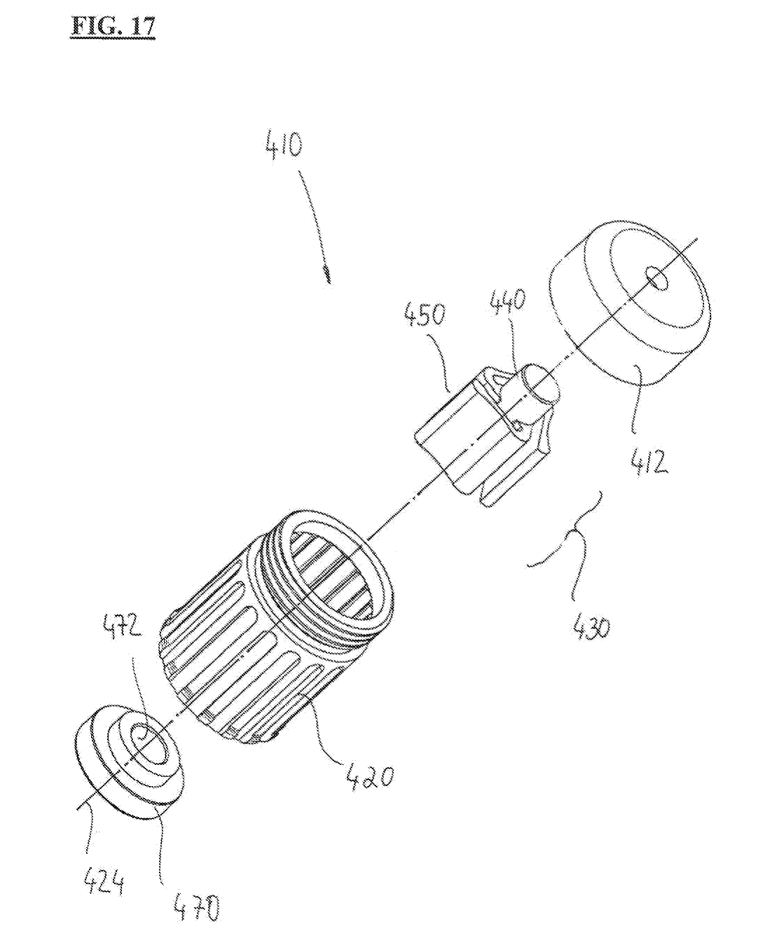

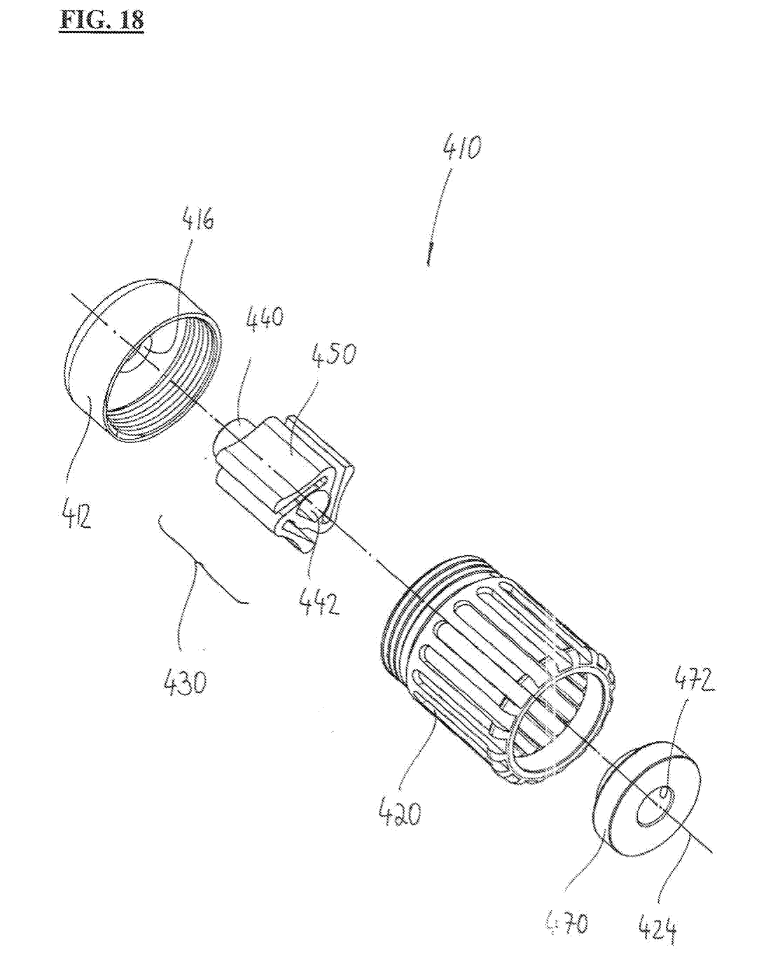

Reference is made to FIG. 16 to 18. FIG. 16 is view in perspective, from two different angles (one with a partial cross section), of an example of a different configuration, a fourth embodiment —410, of a device in accordance with the invention. Similarly to device 310 that was described above when referring to FIG. 11 to 15, also device 410 has a threadable cover 412 and coupler assembly 430 that is extractable and removable by sliding from the house component 420. But with contra distinction to device 310, device 410 does not comprise an integral elongated shank but rather a connector means 442 that enables the mounting of a variety of shank components (not illustrated), in a replaceable manner, and that are suited on their side, each one, to connect with a threadable component (not illustrated) for fastening it by actuating a torque on it. FIGS. 17 and 18 are exploded views in perspective from two different angles, of the components of device 410.

Similarly to device 310 that was described above while referring to FIGS. 11 to 15, also device 410 comprises similar house component 420, cover components 412 and disc 470. But to differ from device 310, in device 410 that is depicted in FIGS. 17-18, in coupler assembly 430, multi springy arms means 450, on its one side, is formed with a segment protruding stem 440 suited to connect with the inner surface area of the central opening 416 of cover component 412. On its second side, multi springy arms means 450 is formed with connector means 442 (also in this illustrated example, in accordance with ISO-1797-1). Connector means 442 enables installing on it in a dismountable, removal and exchangeable manner a variety of elongated shank components (not illustrated) wherein the end of each one of them is suited to connect with a threadable component in order to fasten it by actuating a rotational moment on it (for example—shanks whose ends are formed as a means that is one of a group of means (listed above) re device 10 and while referring to FIGS. 8 to 10).

From the instant of installing means 450 within the inner space of house component 420 (see the partial cross section as a part of FIG. 16), and mounting shank (not illustrated) inside connector means 442, then—the end of the shank would protrude from the house. A segment of the shank would pass through central opening 472 that is formed in disc 470 and the central opening will provide bearing to the coupler assembly.

Thus, in view of the examples that were described above, while referring to the accompanying figures, the following conclusions are mandatory obtained—
a. A rotatable fastening device with an integral torque limiter in accordance with the invention enables the dentist to actuate at will a limited and accurate rotational moment on threaded components that he exploits for treatment inside the patient mouth's limited space.
b. The construction of the device in accordance with the invention is relatively simple. The device is assembled (made up) from a small number of components (see in the illustrated examples—only three (3) to five (5) components).
c. A device in accordance with the invention is given to rinsing and cleaning occasionally, at will, due to the open construction or at least easily accessibility of the device's coupler assembly.
d. A device in accordance with the invention is compact (small dimensions) as dictated by the geometry of the work environment (mouth inner space) and limits the level of the rotational moment (torque) transferred by it in a manner that renders it fail safe, namely—independent of the dentist visual control and with no requirements from the dentist of precise feedback as to the amount of torque he is applying on the fastening device.
e. A device in accordance with the invention can be manufactured as a modular unite in a manner that enables its successful adaptation in advance to the specific dental implant to be fastened, the screw head shape and the specific patient. In other words—the invention enables to provide (instill) to the dentist a large variety of fastening tools that are tailored in advance to the specific procedure to be used (for example—fastening screws, implanting). A device in accordance with the invention provides accurate torque threshold values and those accurate threshold values are fixed (set) at the equipment make level. Namely—at the professional level, those who are experts as to the required torque levels that are needed for optimized fastening of the threadable elements is the manufacture of the devices, in accordance with the invention they provide reproducibility—an additional similar device would also be produced by the same make with the same transferable torque threshold level (a torque value decided at the manufacturing level to optimize the fastening, e.g.—of a specific dental implant). While providing devices with precise torque levels, the manufacture of devices in accordance with the invention, can provide to the dentist a variety of devices intended for the same procedure but each with different pre-defined torque level (in a manner that was weighted in advance—in the manufacture level, the different situations in which different patients might be).
f. A device in accordance with the invention is also liable to be manufactured as a replaceable module that is suited for being installed at the end of a universal tool that serves to provide the rotational moment.

Having described above several aspects of at least one embodiment, it is to be appreciated that various alterations, modifications, and improvements will readily occur to those skilled in the art. Such alterations, modifications, and improvements are intended to be part of this disclosure and are intended to be within the scope of the invention. Accordingly, the foregoing description and drawings are by way of example only, and the scope of the invention should be determined from proper construction of the appended claims, and their equivalents.

1. A rotatable fastening device with an integral torque limiter, comprising:
an house component that is formed as a cylindrical sleeve having an inner space with a central axis that extends along its length, the house component comprising an array of alternating recesses and protrusions resembling inner teeth that is formed inside the house component wherein the array of alternating recesses and protrusions extends around an inner wall circumference of said inner space; and—
a coupler assembly adapted to be included within said house component along the lengthwise axis of said house component, the coupler assembly being configured to connect with a threadable component for fastening said threadable component by actuating a moment on said threadable component, and wherein—
said coupler assembly comprises a means having at least one springy arm, wherein from the instant of installation of said at least one springy arm within said inner space of the house component, a tip of said springy arm is biased to be integrated into a recess of said recesses and protrusions array of said house component; and—
wherein said house component is configured to be actuated by providing a rotatable moment on the house component around said lengthwise axis of said house component, in a manner that—
a. as long as the actuated torque being actuated is at a level that does not exceed a pre-set threshold level, said at least one springy arm tip remains integrated within a recess of said recesses and protrusions array of the house component, thereby said at least one springy arm transfers the torque for turning the coupler assembly concurrently with a rotation of said house component, around longitudinal axis of house component, thereby fastening the threadable component to which said threadable component is connected; and
b. when said actuated torque is at a level that exceeds said preset threshold level, said at least one springy arm is biased into bending so that said tip of said at least one springy arm is moved unto sinking and is extracted from said recess of said recesses and protrusions array of said house component and pushed unto a sliding movement on the surface area of said array, in a manner that the torque transfer is stopped and the continued rotation of said house component around said lengthwise axis of the house component does not lead into a parallel rotation of said coupler assembly and therefore, the fastening of the threadable component to which said threadable component is connected is stopped.

2. A rotatable fastening device with an integral torque limiter in accordance with claim 1, wherein—
said means endowed with having at least one springy arm is a multi springy arms means, wherein all said springy arms extend in a circumferential array while said springy arms protrude from said longitudinal axis of said house component, and the tips of said springy arms are biased to integrate inside a number of recesses in accordance with the number of said springy arms in said recesses and protrusions array of said house component; and—
wherein said house component is configured to be actuated by a rotational moment on the house component around said longitudinal axis of said house component, in a manner that—
a. as long as the actuated torque being actuated is at a level that does not exceed a pre-set threshold level, each tip of all the springy arms remain integrated within a recess of said recesses and protrusions array and all the springy arms transfer the moment for rotating said coupler assembly concurrently with a rotation of said house component around the longitudinal axis of said house component, thereby fastening said threadable component unto which said threadable component is connected; and—
b. when said actuated torque is at a level that exceeds said preset threshold level, all said springy arms are biased into bending in a manner that they are pushed into sinking and are extracted from said recess of said recesses and protrusions array of said house component and pushed unto a sliding movement on the surface area of said array, in a manner that said moment transfer is stopped and the continuation of rotation of said house component around said lengthwise axis of the house component does not lead now into a parallel rotation of said coupler assembly and therefore, said fastening of the threadable component to which said threadable component is connected is stopped.

3. A rotatable fastening device with an integral torque limiter in accordance with claim 2, wherein—
said multi springy arms means is formed with three springy arms that are located in equal distribution around and in relation to said lengthwise axis and each one of said springy arms is formed as a kind of a spring leaf with a rectangular configuration and extends in an essentially tangential direction in relation to said lengthwise axis as an outstretched beam wherein said house component is harnessed to a one end while the other end is free and located at a tip of a forearm sector that can be bent in a movement around said opposing harnessed end.

4. A rotatable fastening device with an integral torque limiter in accordance with claim 2, wherein—
said house component comprises a bracket that is formed inside said house component wherein said bracket extends around the circumference of said inner wall; and—
said coupler assembly comprises a disc component that is located along said multi springy arms means and wherein said disc component serves as a bearing for rotation around said longitudinal axis, together with said multi springy arms means through said bracket that is formed in said house component.

5. A rotatable fastening device with an integral torque limiter in accordance with claim 4, wherein—
said coupler assembly is formed with an elongated shank whose end is suited to connect with said threadable component for fastening said threadable component by actuating a moment on said threadable component, and wherein from an instant of installing said multi springy arms means of said coupler assembly inside said inner space of said house component, said elongated shank end protrudes from said house component; and—
said disc component is suited to be affixed on a segment of said elongated shank of said coupler assembly.

6. A rotatable fastening device with an integral torque limiter in accordance with claim 4, wherein—
said elongated shank and said multi springy arms means are formed as one consolidated homogenous part.

7. A rotatable fastening device with an integral torque limiter in accordance with claim 4, wherein—
said disc component on one side of the house component is connected to multi springy arms means via a connecting shank that extends along said elongated axis and on a second side of the house component formed with a connector means that enables installing on the connector means elongated shanks components, in a dismountable, removal and replaceable manner, where an end of each of the elongated shanks components are configured to connect with said threadable component for fastening said threadable component by actuating a moment on said threadable component.

8. A rotatable fastening device with an integral torque limiter in accordance with claim 7, wherein—
said disc component, connecting shank and multi springy arms means are formed as a consolidated one homogenous part.

9. A rotatable fastening device with an integral torque limiter in accordance with claim 7, wherein—
said connector means that enables installing elongated shank components in a dismountable, removal and replaceable manner, is a connector adapted to ISO 1791-1.

10. A rotatable fastening device with an integral torque limiter in accordance with claim 4, wherein—
said house component is formed with an intermediate segment that is formed inside said house component where the intermediate segment extends around a circumference of said inner space while the intermediate segment protrudes from said surface of said inner wall and separates between said alternating recesses and protrusions array and said bracket; and—
wherein assembling the rotatable fastening device requires pushing said multi springy arms means by exerting force, from the direction of said bracket to movement in the one direction—along the lengthwise axis of house component towards and beyond said intermediate segment; until connecting of said disc surface with a one side of said intermediate segment, and;—
wherein disassembly of said rotatable fastening device necessitates the pushing of said multi springy arms means, from a direction of said array of said alternating recesses and protrusions—into movement in the other opposite direction—along said longitudinal axis of the house component, to said other side of said intermediate segment while passing over said intermediate segment and extracting said disc component from said bracket.

11. A rotatable fastening device with an integral torque limiter in accordance with claim 10, wherein—
said intermediate segment is formed on one side of the house component that is connected with said disc component surface as a protruding shoulder and on a second side of the house component that is connected with said multi springy arms means as a slanted depression.

12. A rotatable fastening device with an integral torque limiter in accordance with claim 2, wherein—
said house component comprises a bracket that is formed inside said house component wherein said bracket extends around said inner space circumference on said inner wall, and on said second end of said house component is formed with an external thread portion formed on a surface segment from said outer surface of said house component; and—
additionally said rotatable fastening device comprises also—
a. a disc component endowed with a central opening and suited to be affixed inside said bracket that is formed within said house component and to provide bearing of said coupler assembly for turning around said lengthwise axis via said inner surface area of said central opening of said disc component; and—
b. a cover component formed with an internal thread portion that matches said external thread portion of said house component and with a central opening, and wherein said cover component is suited to be mounted by threading to said house component and also to provide a bearing to said coupler assembly for turning around said longitudinal axis by said inner surface of said central opening of said disc component.

13. A rotatable fastening device with an integral torque limiter in accordance with claim 12, wherein—
said coupler assembly is formed with an elongated shank that protrudes on the two sides of said multi springy arms means; and—
a first end of said elongated shank configured to connect with said threadable component for said threadable component, and from the instant of installing said multi springy arms means of said coupler assembly inside said inner space of said house component, one end of said elongated shank protrudes from said house component; and—
a segment of said elongated shank between said multi springy arms means and said first end of said elongated shank is configured to connect for contact with the inner surface of said central opening of said disc component; and—
a second end of said elongated shank is configured to connect with the inner surface area of said central opening of said cover component.

14. A rotatable fastening device with an integral torque limiter in accordance with claim 13, wherein—
said elongated shank and said multi springy arms means are two separate components, and wherein said multi springy arms means is formed with a pass through opening that is suited for affixing said shank therein while said shank protrudes from two sides of the pass through opening.

15. A rotatable fastening device with an integral torque limiter in accordance with claim 13, wherein—
said elongated shank is formed with a protruding shoulder that is suited to connect for contact with surface area of said disc component in a manner that prevents the sliding of said shank through said disc's opening; and—
said cover component is formed with a shoulder protruding from said inner surface area of said central opening of the cover component and is suited for contact with said elongated shank's end in a manner that prevents sliding of said shank through said central opening.

16. A rotatable fastening device with an integral torque limiter in accordance with claim 12, wherein—
said multi springy arms means on one side is formed with a protruding stem segment that is suited to contact with said inner surface area of said central opening of said cover component; and—
on a second side of said multi springy arms means, formed with a connector means that enables installing elongated shank components on said connector means, in a dismountable, removal and replaceable manner and wherein said end of each of said elongated shank components is suited to contact with said threadable component for fastening said threadable component by actuating a moment on said threadable component; and—
wherein from the instant of installing said multi springy arms means inside said inner space of said house component, said shank's end protrudes from said house component; and—
a segment of said elongated shank is suited for contact with the inner surfaces of said central opening of said disc component.

17. (canceled)

18. A rotatable fastening device with an integral torque limiter in accordance with claim 5, wherein—
said end of said elongated shank that is adapted to connect with said threadable component for fastening said threadable component by actuating a moment on said threadable component is formed as a means selected from a group consisting of: a tip key, an end of a hexagonal screws key 0.05″, an implant's hexagonal 2.45 mm key, a specific designated key including a conic connections key for implants of a selected make or another, in a narrow standard or a wide platform, a screwdriver head, and a socket spanner.

19. A rotatable fastening device with an integral torque limiter in accordance with claim 1, wherein—
said house component is formed on outer surfaces of the rotatable fastening device with means that enables connecting the rotatable fastening device with a ratchet key and actuating with said ratchet key a moment on said rotatable fastening device.

20. A rotatable fastening device with an integral torque limiter in accordance with claim 19, wherein—
said means that enables connection of the rotatable fastening device with a ratchet key, comprises an array of longitudinal recesses and protrusions that extend alternating on at least a segment of said house component and around said outer circumference of said house component.

21. A rotatable fastening device with an integral torque limiter in accordance with claim 20, wherein—
said means that enables connection of said rotatable fastening device with a ratchet key, comprises in addition a circumferential shoulder that is formed alongside said array of longitudinal recesses and protrusions wherein said circumferential shoulder protrudes from said house component in order to prevent sliding of said ratchet key from the instant of connection of said ratchet key with said array.
